(19) United States
(12) Patent Application Publication
(10) Pub. No.: US 2008/0014573 A1
(43) Pub. Date: Jan. 17, 2008

(54) Biosample Manipulation Apparatus

(75) Inventor: Toshihiko Nagamura, Osaka (JP)

Correspondence Address: KRATZ, QUINTOS & HANSON, LLP, 1420 K Street, N.W., Suite 400, WASHINGTON, DC, 20005, US

(73) Assignee: YUNISOKU CORPORATION, HIRAKATA-SHI, OSAKA (JP)

(21) Appl. No.: 11/596,579

(22) PCT Filed: May 26, 2004
(86) PCT No.: PCT/JP04/07170

§ 371 Date: Nov. 15, 2006

(51) Int. Cl. C12Q 1/00

(52) U.S. Cl. 435/4

(57) ABSTRACT

A biosample manipulation method according to the invention includes the steps of: providing a scanning probe microscope (3) with a cantilever (9) including a beam portion (15) with its base end supported in a cantilever manner, a probe (17) formed to protrude from the beam portion (15) and having a hollow (16) in itself, a fine hole (21) formed to penetrate from the hollow (16) to an outside of the probe (17) through the probe (17), and a first electrode (20) provided in the hollow (16): filling fluid (19) including a physiologically active substance or a substance for exciting a chemical reaction and having conductivity in the hollow (16) in the cantilever (9); placing a biosample (2) on a second electrode (12) formed of a conductive thin film: bringing the probe (17) of the cantilever (9) close to the biosample (2); applying a pulse voltage between the first electrode (20) and the second electrode (12) to thereby allow the fluid (19) to flow out of the fine hole (21) and inject the fluid (19) into the biosample (2); and observing change in a surface profile of the biosample (2) through the scanning probe microscope.

### TECHNICAL FIELD

The present invention relates to a biosample manipulation apparatus for injecting or dropping a medicinal solution including a gene, protein, an enzyme, or the like into or on a biosample such as a cell and particularly to a biosample manipulation apparatus using a cantilever.

### BACKGROUND TECHNIQUE

Conventionally, in a field of biological research, pathologic research, or the like, a gene or the like is injected into and expressed in a cell. Here, as a means for injecting the gene or the like into a biosample such as a cell, a method has been conventionally proposed using a cantilever provided to a scanning probe microscope (see Patent Document 1). FIG. 8 is a drawing showing an example of a prior-art biosample manipulation apparatus. As shown in the drawing, the biosample manipulation apparatus 80 includes a cantilever 82 provided to a scanning probe microscope (not shown) and protruding a probe 81 in a sharp-pointed shape from a tip end portion of the cantilever 82 and a needle-like object 83 attached to a tip end of the cantilever 82 and formed of a carbon nanotube or the like. In using this biosample manipulation apparatus 80, a gene or the like is fixed to a tip end of the needle-like object 83, the cantilever 82 is caused to scan to insert the needle-like object 83 into a cell 84 to thereby introduce the gene or the like into the cell 84, and this state is retained to express the gene.

On the other hand, the present applicant has conventionally proposed a micro-machining apparatus using a cantilever (see Patent Document 2). This micro-machining apparatus includes a cantilever portion supported in a cantilever manner and a protruding portion formed to protrude from a tip end of the cantilever portion. A hollow is formed in the protruding portion and a fine hole leading outside the protruding portion from the hollow is formed to penetrate the protruding portion. Here, in the hollow in the protruding portion, fluid such as indium, gallium, an alloy of indium and gallium, or the like is filled. In using the micro-machining apparatus, by application of a pulse voltage to the cantilever, the fluid flows outside through the fine hole to form an ultrafine dot or a ultrafine line of In, Ga, or the like on a surface of a sample. In this way, a semiconductor light emitting device or the like for high-speed optical communication is produced.

However, in the prior-art biosample manipulation apparatus 80, it is necessary to fix the gene or the like to be introduced into the cell 84 to the tip end of the needle-like object 83 and therefore there is a problem that a medicinal liquid in which a gene, a chemical, or the like is dissolved or fluid such as a reactive gas cannot be injected or dropped into the cell 84.

Moreover, since the needle-like object 83 having a diameter of about 10 nm to 30 nm is inserted into the cell, a hole 86 greater than the diameter of the needle-like object 83 is formed in a cell membrane 85 or the needle-like object 83 may damage a cell nucleus 87 in some cases. As a result, there are also problems that the cell 84 may suffer great damage and die or it takes the cell 84 a long time to repair the hole 86 or damage by its self-repairing function.

Patent Document 1: Japanese Patent Application Laid-open No. 2003-325161

Patent Document 2: Japanese Patent Application Laid-open No. 2004-34277

### DISCLOSURE OF THE INVENTION

The present invention has been made with the above problems in view and it is an object of the invention to provide a means for injecting or dropping fluid such as medicinal liquid in which a gene or the like is dissolved into a biosample while minimizing damage caused to the biosample at this time.

According to the invention, a biosample manipulation apparatus for injecting a physiologically active substance or a substance for exciting a chemical reaction into a biosample includes: a beam portion with its base end supported in a cantilever manner; a probe formed to protrude from a tip end of the beam portion and having a hollow in itself a fine hole formed to penetrate from the hollow to an outside of the probe through the probe; fluid filled in the hollow; and a fluid injecting means for allowing the fluid to flow out of the fine hole and injecting the fluid into the biosample. According to the invention, the fluid injecting means can allow a minute amount of fluid filled in the hollow in the probe to flow out of the fine hole and inject the fluid into the biosample.

Moreover, according to the invention, the fluid injecting means includes: a first electrode provided in contact with the fluid; a second electrode provided in contact with the biosample; and a pulsed power supply for applying a pulse voltage between the first electrode and the second electrode. According to the invention, by applying the pulse voltage to the fluid, binding force acting between respective particles forming the fluid is cut off and the fluid that has lost cohesion flows out of the fine hole. By applying the pulse voltage to the biosample, a hole is formed in a surface of the biosample due to a transitory dielectric breakdown and the fluid passes through the hole and is taken into the biosample due to electrophoresis. Since the hole is extremely minute and closes up soon due to a self-repairing function of the biosample, the biosample does not suffer much damage. In this way, it is possible to inject the fluid into the biosample while minimizing the damage caused to the biosample.

Furthermore, according to the invention, the biosample manipulation apparatus for dropping a physiologically active substance or a substance for exciting a chemical reaction on a biosample includes: the beam portion with its base end supported in the cantilever manner; the probe formed to protrude from a tip end of the beam portion and having a hollow in itself; a fine hole formed to penetrate from the hollow to an outside of the probe through the probe; fluid filled in the hollow; and a fluid outflow means for allowing the fluid to flow out of the fine hole. According to the invention, the fluid outflow means can allow a minute amount of fluid filled in the hollow in the probe to flow out of the fine hole and drop the fluid on the biosample.

Moreover, according to the invention, the fluid outflow means includes a pair of electrodes provided in contact with the fluid and the pulsed power supply for applying a pulse voltage between the respective electrodes. According to the invention, by applying the pulse voltage to the fluid, binding force acting between respective particles forming the fluid is cut off and the fluid flows out of the fine hole.

Furthermore, according to the invention, the beam portion, the probe, and the fine hole constitute a probe of a scanning probe microscope. According to the invention, after manipulation of the biosample, it is possible to check if a medicinal liquid or the like has been injected into or dropped on the biosample reliably and a degree of breakage of the biosample due to application of the pulse voltage.

Moreover, the invention comprises a manipulation observing means for observing manipulation of the biosample and change of the biosample after the manipulation. According to the invention, it is possible to easily select a biosample to be manipulated from a plurality of arranged biosamples and to manipulate the biosample more accurately while observing through the manipulation observing means. It is also possible to observe the inner change occurring in the biosample after the manipulation.

### BRIEF DESCRIPTION OF THE DRAWINGS

FIG. 1 is a diagrammatic illustration of a biosample manipulation apparatus 1, 30, 40, 50, or 60 according to an embodiment of the invention.

FIG. 2 is a partially enlarged sectional view of a vicinity of a cell 2 in FIG. 1.

FIG. 3 is a schematic perspective view of a structure of a cantilever 9 according to a first embodiment.

FIG. 4 is a schematic perspective view of a structure of a cantilever 31 according to a second embodiment.

FIG. 5 is a schematic perspective view of a structure of a cantilever 41 according to a third embodiment.

FIG. 6 is a schematic perspective view of a structure of a cantilever 51 according to a fourth embodiment.

FIG. 7 is a schematic perspective view of a structure of a cantilever 61 according to a fifth embodiment.

FIG. 8 is a schematic side view of a prior-art biosample manipulation apparatus 80.

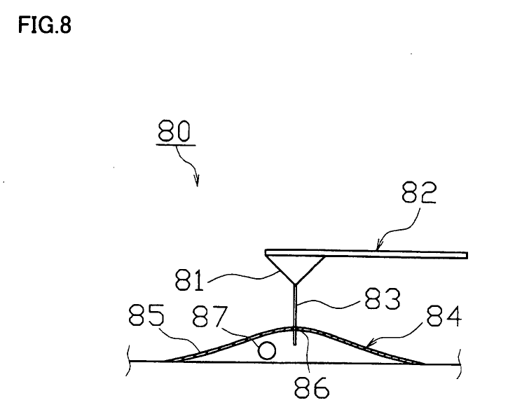

FIG. 9 is an attached sheet for explaining reference numerals used in the respective drawings.

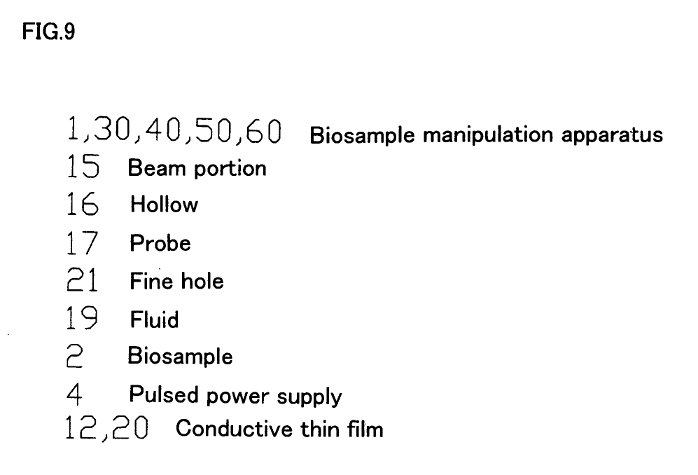

### BEST MODES FOR CARRYING OUT THE INVENTION

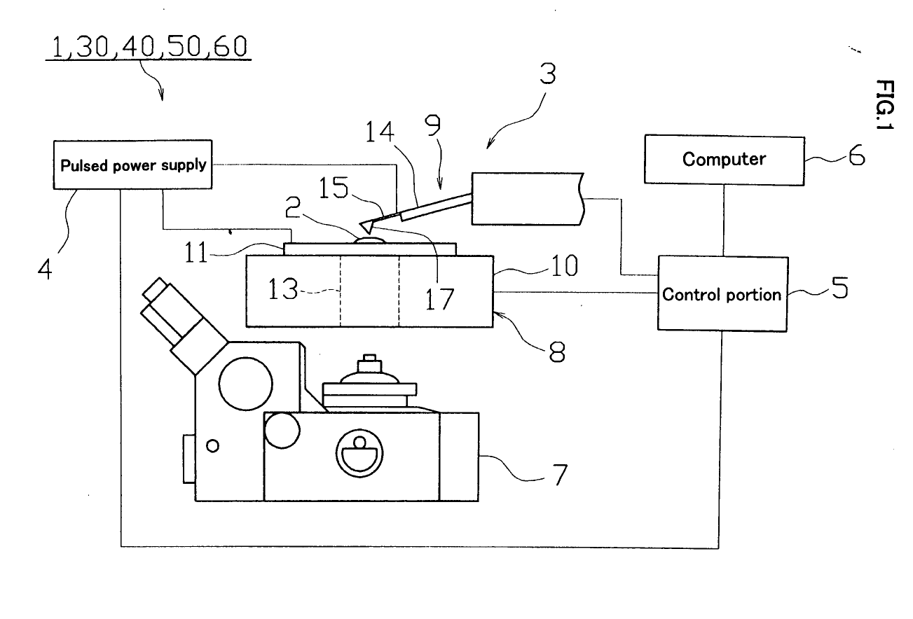

A first embodiment of the present invention will be described below with reference to the drawings. FIG. 1 is a diagrammatic illustration of a biosample manipulation apparatus 1 according to the present embodiment. As shown in the drawing, the biosample manipulation apparatus 1 includes an atomic force microscope (hereafter referred to as “AFM”) 3 for manipulating a cell (biosample) 2 as an object of manipulation and for observing change of a surface of the cell 2, a pulsed power supply 4 for applying a pulse voltage to the AFM 3, a control portion 5 for controlling operations of the AFM3 and the pulsed power supply 4, a computer 6 which is an input/output portion of the control portion 5, and an optical microscope (manipulation observing means) 7 for observing the cell manipulation and inner changes of the cell 2 after the manipulation. Although the cell 2 is taken as an example of the biosample in description of the embodiment, it is also possible to use a piece of tissue of a living thing or a biopolymer such as a protein and an enzyme besides the cell.

The AFM3 is a type of a scanning probe microscope and is for observing a surface profile of the cell 2 and manipulating the cell 2. As shown in FIG. 1, the AFM3 includes a scanning stage 8 on which the cell 2 is placed and a cantilever 9 for scanning a surface of the cell 2 on the scanning stage 8.

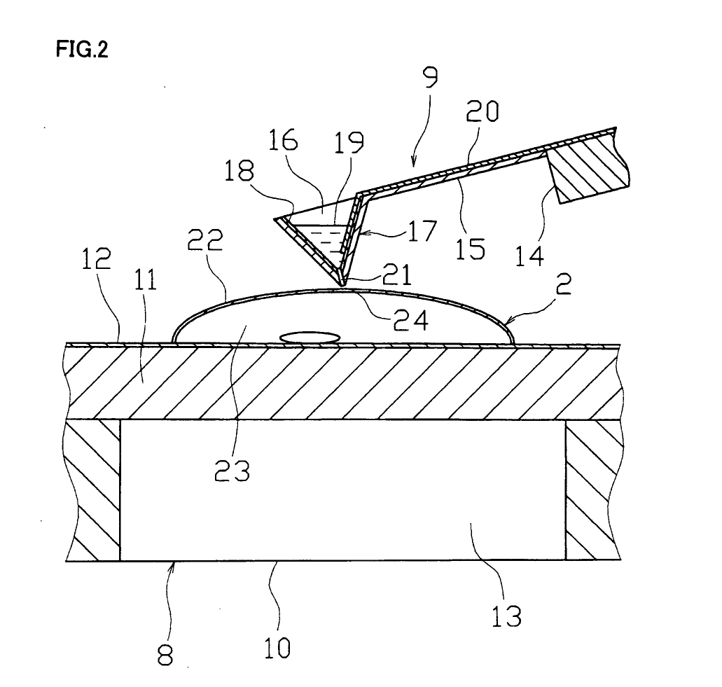

FIG. 2 is a partially enlarged sectional view of a vicinity of a cell 2 in FIG. 1. As shown in FIGS. 1 and 2, the scanning stage 8 includes a stage main body 10 in which a piezoelectric device actuator (not shown) is mounted, a glass plate 11 disposed on an uppermost portion of the stage main body 10, and a transparent conductive thin film (second electrode) 12 stuck on an upper face of the glass plate 11. In a state in which the cell 2 is placed on the conductive thin film 12, it is possible to move the cell 2 in directions of X, Y, and Z axes by applying a voltage to the piezoelectric device actuator to expand and contract the scanning stage 8. As shown in FIG. 1, a cavity 13 vertically penetrating the stage main body 10 is formed in the stage main body 10 and the optical microscope 7 is disposed below the cavity 13. In this way, through the glass plate 11 and the conductive thin film 12, it is possible to observe a manner of manipulation of the cell 2, changes in the cell 2, and the like through the optical microscope 7.

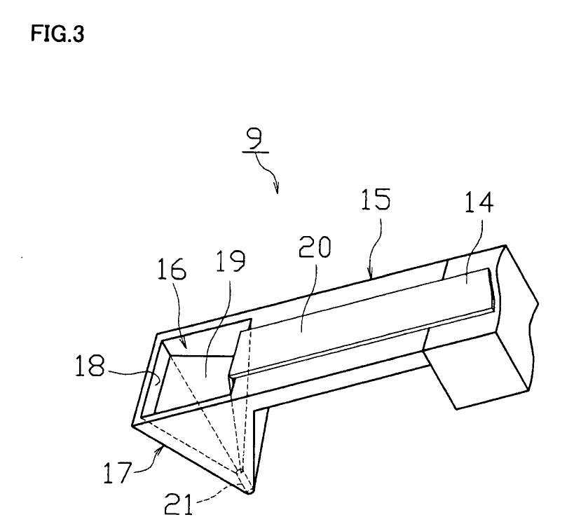

FIG. 3 is a schematic perspective view of a structure of a cantilever 9. As shown in FIGS. 1 to 3, the cantilever 9 includes a beam portion 15 with its base end supported in a cantilever manner by a holder 14, a probe 17 formed to protrude from a tip end of the beam portion 15 and having a hollow 16, a mask 18 applied to an inner wall face of the probe 17, medicinal liquid 19 filled in the hollow 16, a conductive thin film (first electrode) 20 provided to extend from a surface of the beam portion 15 to the inner wall face of the probe 17, and a fine hole 21 formed to penetrate the probe 17 from the hollow 16 to an outside of the probe 17.

The probe 17 comes extremely close to the surface of the cell 2 to detect a surface profile of the cell 2. The probe 17 is in a shape of a quadrangular pyramid and the hollow 16 is formed to extend from a bottom face side toward a vertex side of the quadrangular pyramid in a shape of a quadrangular pyramid as shown in FIG. 3. The mask 18 lowers surface tension of the medicinal liquid 19 to thereby make the medicinal liquid 19 liable to flow outside through the fine hole 21. In the embodiment, the thin film is formed by applying gold to the inner wall face of the probe 17. In the invention, the mask 18 is an arbitrary component and ingredients, a film thickness, and the like of the mask 18 can be properly changed in design. The medicinal liquid 19 is prepared by dissolving a nucleic acid substance such as DNA or a protein substance such as an antigen, an enzyme, and a hormone (hereafter they will be referred to as “physiologically active substances”) in a buffer solution and has conductivity. Since an opening diameter of the fine hole 21 is extremely minute and respective particles forming the medicinal liquid 19 are bound together by action of atomic force or the like, this medicinal liquid 19 is retained in the probe 17 without flowing outside the probe 17 through the fine hole 21. The medicinal liquid 19 may be one exciting a chemical reaction when a minute amount of the liquid is dropped on the cell 2 besides the liquid in the embodiment. The fine hole 21 functions as an outlet for allowing the medicinal liquid 19 filled in the hollow 16 to flow outside the probe 17. The fine hole 21 is an ultrafine hole having a circular cross section and an opening diameter of 20 to 500 nm. The fine hole 21 is formed from a vertex of the hollow 16 toward a vertex of the probe 17 as shown in FIG. 3. Therefore, the vertex of the probe 17 is chipped and the tip end of the probe 17 is formed of a section of the fine hole 21. By bringing this section extremely close to the surface of the cell 2, the surface profile is scanned. By changing a size of the cross section of the fine hole 21, it is possible to adjust an amount of outflow of the medicinal liquid 19. The conductive thin film 20 is for applying the pulse voltage to the medicinal liquid 19 and the cell 2. This conductive thin film 20 is stuck on an upper face of the beam portion 15 in a vertical direction of the beam portion 15 and a tip end portion of the film 20 is bent down and stuck along the inner wall face of the probe 17 as shown in FIGS. 2 and 3. Therefore, when the medicinal liquid 19 is filled in the hollow 16, the tip end portion of the conductive thin film 20 is in contact with the medicinal liquid 19.

The pulsed power supply 4 is for applying the pulse voltage to the medicinal liquid 19 and the cell 2. This pulsed power supply 4 is connected to the conductive thin film 20 of the cantilever 9 and the conductive thin film 12 of the scanning stage 8, respectively, and applies the pulse voltage between the conductive thin film 20 and the conductive thin film 12 as shown in FIGS. 1 and 2. Here, since the medicinal liquid 19 has conductivity and a cell membrane 22 and cell fluid 23 forming the cell 2 also have conductivity, a current passes between the conductive thin film 20 and the conductive thin film 12. At this time, the binding force acting between the respective particles forming the medicinal liquid 19 is cut off due to reception of an electric shock and the medicinal liquid 19 that has lost cohesion flows outside the probe 17 through the fine hole 21. The amount of outflow of the medicinal liquid 19 can be adjusted by controlling magnitude of the pulse voltage. Moreover, since the current also flows through the cell 2, a fine breakdown hole 24 is formed in the cell membrane 22 due to a transitory dielectric breakdown and the medicinal liquid 19 electrophoresed by the pulse voltage passes through the breakdown hole 24 in the cell membrane 22 and is taken into the cell 2. Since the breakdown hole 24 formed in the cell membrane 22 is extremely fine, the hole 24 closes up soon due to the self-repairing function of the cell 2 and the cell 2 does not suffer much damage. In this way, the medicinal liquid 19 filled in the hollow 16 is injected into the cell 2. Although the medicinal liquid 19 flowing out through the fine hole 21 is injected into the cell 2 in the embodiment, the embodiment is not limited to this. It is also possible to drop the flowing-out medicinal liquid 19 on the cell 2. In this case, though it is not shown in detail in the drawing, if a pair of electrodes is provided in contact with the medicinal liquid 19 and a pulse voltage is applied between the respective electrodes, a dielectric breakdown does not occur in the cell 2 and the flowing-out medicinal liquid 19 is dropped on the cell 2 without being taken into the cell 2. As a means for cutting off the binding force acting between the respective particles of the medicinal liquid 19, besides application of the electric shock to the medicinal liquid 19 as in the embodiment, for example, a method in which a mechanical shock is applied by a pulse laser may be used.

The control portion 5 controls the operations of the AFM 3 and the pulsed power supply 4. The control portion 5 causes the scanning stage 8 to scan in horizontal directions, i.e., the directions of the X and Y axes while controlling the scan in the direction of the Z axis, i.e. a vertical direction so that the atomic force acting between the probe 17 of the cantilever 9 and the cell 2 becomes constant. At this time, a feedback amount in the direction of the Z axis corresponding to a position in the directions of the X and Y axes of the scanning stage 8 is detected as an output voltage of the control portion 5 and is output as a three-dimensional image on a screen through an arithmetical unit (not shown) in the computer 6 to thereby measure the surface profile of the cell 2 highly precisely. In the embodiment, the scanning stage 8 is moved to scan the surface of the cell 2 while fixing the position of the cantilever 9. On the contrary, it is also possible to move the cantilever 9 to scan the surface of the cell 2 while fixing the position of the scanning stage 8. In this case, the control portion 5 may control the operation of the cantilever 9. The control portion 5 also controls the operation of the pulsed power supply 4. While causing the scanning stage 8 to scan to observe the surface of the cell 2, it is possible to apply the voltage pulse to inject or drop the medicinal liquid 19 into or on the cell 2 when the cantilever 9 has reached a desired position. Although the case where one cell 2 is placed on the scanning stage 8 is described as an example in the embodiment, it is also possible to arrange a plurality of cells 2 on the scanning stage 8 and to inject or drop the medicinal liquid 19 only into or on an arbitrary cell 2 selected from the cells 2. After injection of the medicinal liquid 19 into the cell 2 in this manner, it is possible to observe the surface profile of the cell 2 by using the AFM3 to check if the medicinal liquid 19 has been injected into the cell 2 reliably and a degree of breakage of the cell 2 due to application of the pulse voltage, which allows more accurate cell manipulation.

A procedure of the cell manipulation by using the biosample manipulation apparatus 1 will be described below. First, predetermined medicinal liquid 19 to be injected into or dropped on the cell 2 is filled in the hollow 16 in the cantilever 9. Next, the cell 2 to be manipulated is placed on the scanning stage 8 and positioning is carried out to bring the probe 17 of the cantilever 9 right above the cell 2 while observing through the optical microscope 7. Then, after the scanning stage 8 is caused to scan in the direction of the Z axis to bring the probe 17 close to the cell 2, the scanning stage 8 is caused to scan in the directions of the X and Y axes. When the probe 17 has reached a desired position, the pulsed power supply 4 is operated to apply a pulse voltage of predetermined magnitude between the respective conductive thin films 12 and 20. As a result, the medicinal liquid 19 flows out of the fine hole 21 of the cantilever 9 and is injected into or dropped on the cell 2. Then, change in the surface profile of the cell 2 is observed through the AFM3 and change occurring in the cell 2, e.g., a manner of expression of the gene injected into the cell 2 is observed through the optical microscope 7.

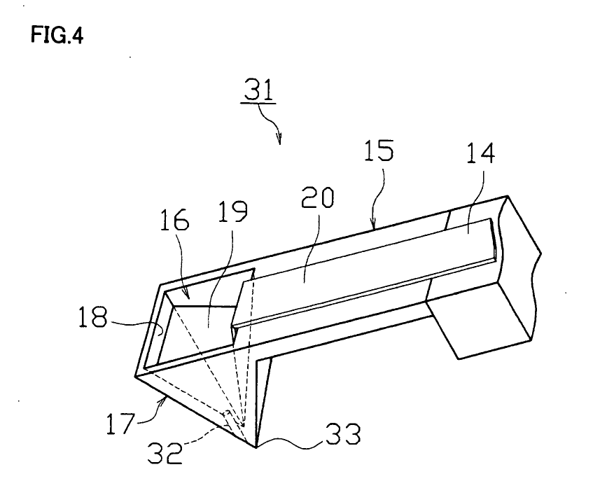

Next, a second embodiment of the invention will be described with reference to the drawing. A biosample manipulation apparatus 30 according to the present embodiment is characterized in that it is different in a structure of a cantilever 31 from the biosample manipulation apparatus 1 in the first embodiment. Other structures are the same as those in the first embodiment and detailed description of them will be omitted here. FIG. 4 is a schematic perspective view of the structure of the cantilever 31 according to the embodiment. In FIG. 4, the same components as those in FIG. 3 are provided with the same reference numerals. As shown in FIG. 4, the cantilever 31 includes a beam portion 15 with its base end supported in a cantilever manner by a holder 14, a probe 17 formed to protrude from a tip end of the beam portion 15 and having a hollow 16, a mask 18 applied to an inner wall face of the probe 17, medicinal liquid 19 filled in the hollow 16, a conductive thin film 20 provided to extend from a surface of the beam portion 15 to the inner wall face of the probe 17, and a fine hole 32 connecting the hollow 16 and an outside of the probe 17.

In the cantilever 9, the fine hole 32 is not positioned on a line connecting a vertex of the hollow 16 and a vertex of the probe 17 and formed in a position slightly displaced from the line. As a result, the vertex of the probe 17 is not chipped and remains as a probe point 33 in a sharp-pointed shape. This probe point 33 is formed to have a radius of curvature of about 10 nm and therefore can scan the surface of the cell 2 at higher resolution as compared with the cantilever 9 in which the tip end of the probe 17 is formed of the fine hole 21 having the opening diameter of about 20 to 500 nm. Here, in order to allow the medicinal liquid 19 to flow out to a predetermined position designated by the probe point 33 more accurately, it is preferable to form the fine hole 32 in a closer position to the probe point 33.

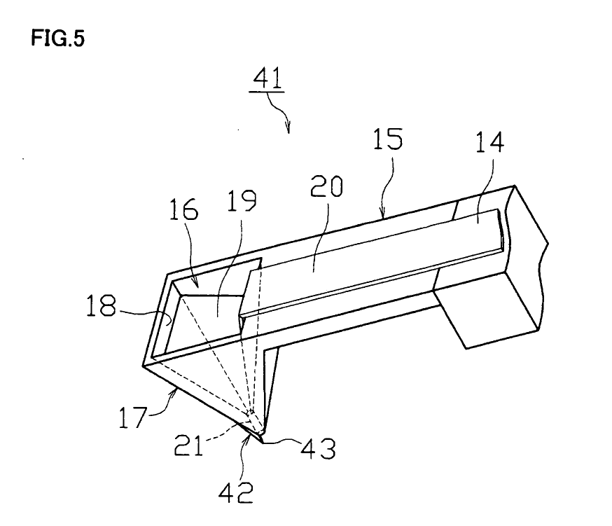

Next, a third embodiment of the invention will be described with reference to the drawing. A biosample manipulation apparatus 40 according to the present embodiment is characterized in that it is different in a structure of a cantilever 41 from the biosample manipulation apparatus 1 in the first embodiment. Other structures are the same as those in the first embodiment and detailed description of them will be omitted here. FIG. 5 is a schematic perspective view of the structure of the cantilever 41 according to the embodiment. In FIG. 5, the same components as those in FIG. 3 are provided with the same reference numerals. As shown in FIG. 5, the cantilever 41 includes a beam portion 15 with its base end supported in a cantilever manner by a holder 14, a probe 17 formed to protrude from a tip end of the beam portion 15 and having a hollow 16, a mask 18 applied to an inner wall face of the probe 17, medicinal liquid 19 filled in the hollow 16, a conductive thin film 20 provided to extend from a surface of the beam portion 15 to the inner wall face of the probe 17, a fine hole 21 connecting the hollow 16 and an outside of the probe 17, and a protruding portion 42 provided to a tip end portion of the probe 17 and functioning as a probe point.

In the cantilever 41, the fine hole 21 is formed from a vertex of the hollow 16 toward a vertex of the probe 17 and the vertex of the probe 17 is chipped similarly to the cantilever 9 in the first embodiment. On the other hand, the protruding portion 42 is formed in a triangular shape having a sharp-pointed corner portion 43 and one end edge of the protruding portion 42 is secured to an outer wall face of the probe 17 so that the sharp-pointed corner portion 43 is positioned below the fine hole 21. By bringing the sharp-pointed corner portion 43 of the protruding portion 42 extremely close to the surface of the cell 2, it is possible to scan the surface of the cell 2 at higher resolution as compared with the cantilever 9 in the first embodiment. It is essential only that the protruding portion 42 have the sharp-pointed corner portion 43 and the shape of the protruding portion 42 is not limited to the triangle but may be changed properly in design.

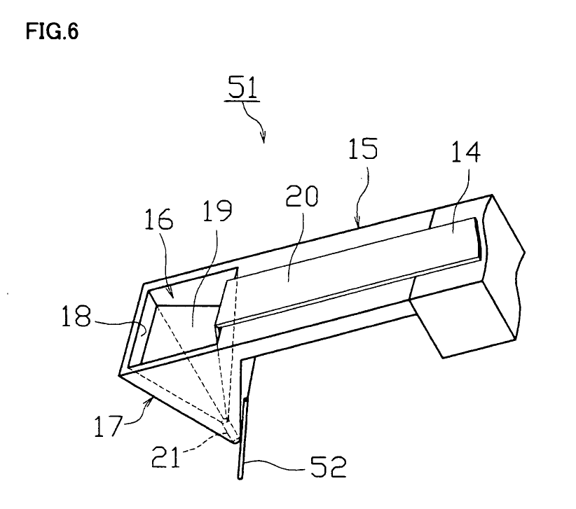

Next, a fourth embodiment of the invention will be described with reference to the drawing. A biosample manipulation apparatus 50 according to the present embodiment is characterized in that it is different in a structure of a cantilever 51 from the biosample manipulation apparatus 1 in the first embodiment. Other structures are the same as those in the first embodiment and detailed description of them will be omitted here. FIG. 6 is a schematic perspective view of the structure of the cantilever 51 according to the embodiment. In FIG. 6, the same components as those in FIG. 3 are provided with the same reference numerals. As shown in FIG. 6, the cantilever 51 includes a beam portion 15 with its base end supported in a cantilever manner by a holder 14, a probe 17 formed to protrude from a tip end of the beam portion 15 and having a hollow 16, a mask 18 applied to an inner wall face of the probe 17, medicinal liquid 19 filled in the hollow 16, a conductive thin film 20 provided to extend from a surface of the beam portion 15 to the inner wall face of the probe 17, a fine hole 21 connecting the hollow 16 and an outside of the probe 17, and a nanotube 52 attached to a tip end portion of the probe 17.

In the cantilever 51, the fine hole 21 is formed from a vertex of the hollow 16 toward a vertex of the probe 17 and the vertex of the probe 17 is chipped similarly to the cantilever 9 in the first embodiment. On the other hand, the nanotube 52 is made of carbon or the like and a base end portion of the nanotube 52 is secured to an outer wall face of the probe 17 so that a tip end portion of the nanotube 52 protrudes below the tip end of the probe 17. An opening diameter of the nanotube 52 is extremely minute. By bringing the tip end of the nanotube 52 extremely close to the surface of the cell 2, it is possible to scan the surface of the cell 2 at higher resolution as compared with the cantilever 9 in the first embodiment.

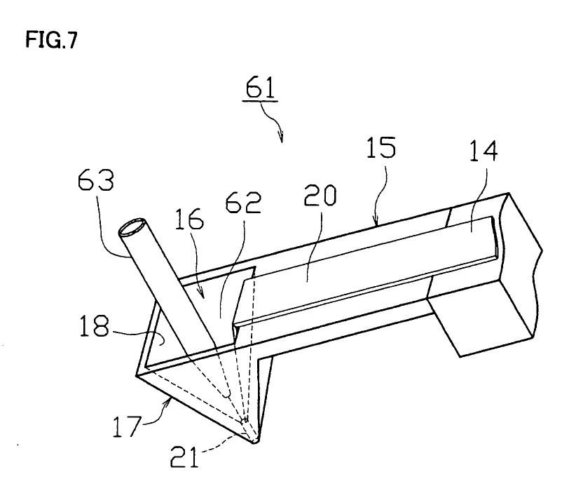

Next, a fifth embodiment of the invention will be described with reference to the drawing. A biosample manipulation apparatus 60 according to the present embodiment is characterized in that it is different in a structure of a cantilever 61 from the biosample manipulation apparatus 1 in the first embodiment. Other structures are the same as those in the first embodiment and detailed description of them will be omitted here. FIG. 7 is a schematic perspective view of the structure of the cantilever 61 according to the embodiment. In FIG. 7, the same components as those in FIG. 3 are provided with the same reference numerals. As shown in FIG. 7, the cantilever 61 includes a beam portion 15 with its base end supported in a cantilever manner by a holder 14, a probe 17 formed to protrude from a tip end of the beam portion 15 and having a hollow 16, a mask 18 applied to an inner wall face of the probe 17, reactive gas (fluid) 62 filled in a tank (not shown), a feed nozzle 63 for feeding the reactive gas 62 to the cantilever 61, a conductive thin film 20 provided to extend from a surface of the beam portion 15 to the inner wall face of the probe 17, and a fine hole 21 connecting the hollow 16 and an outside of the probe 17.

The reactive gas 62 is a generic term used to refer to substances which do not vaporize in a vacuum and which excite various chemical reactions with a surface of a substance and includes compounds, mixtures, and the like as well as elements. As the reactive gas 62, it is possible to use halogen gas represented by HF or HCl, gas cyanide represented by C4H5N or CH3CH2CN, and the like, for example. Besides those, it is possible to use one in a solid or liquid state at room temperature after vaporizing it by heating or the like. Although it is not shown in detail in the drawing, the reactive gas 62 is filled in the tank that is a vacuum chamber. On the other hand, one end of the feed nozzle 63 is connected to the tank and the other end of the nozzle 63 is throttled thin and inserted into the hollow 16. The reactive gas 62 shot from the feed nozzle 63 is not shot outside the probe 17 through the fine hole 21 but stays in the hollow 16 since the opening diameter of the fine hole 21 is extremely minute and respective particles forming the reactive gas 62 are bound together by action of atomic force or the like similarly to the above-described medicinal liquid 19. By application of the pulse voltage to the reactive gas 62 from the conductive thin film 20, the binding force acting between the respective particles is cut off and the reactive gas 62 flows outside the probe 17. In this way, by allowing the reactive gas 62 to flow out of the fine hole 21 and adhere to the surface of the cell 2, various chemical reactions are excited between the cell 2 and the reactive gas 62 and this change is observed by the AFM3 and the optical microscope 7.

### INDUSTRIAL APPLICABILITY

In the invention, not only the cantilever provided to the atomic force microscope but also cantilevers provided to other scanning probe microscopes may be used.

1. A biosample manipulation method for injecting a physiologically active substance or a substance for exciting a chemical reaction into a biosample comprising the steps of:
providing a scanning probe microscope with a cantilever comprising
a beam portion with its base end supported in a cantilever manner, a probe formed to protrude from a tip end of the beam portion and having a hollow in itself, a fine hole formed to penetrate from the hollow to an outside of the probe through the probe, and a first electrode provided in the hollow;
filling fluid including the physiologically active substance or the substance for exciting the chemical reaction and having conductivity in the hollow in the cantilever;
placing the biosample on a second electrode formed of a conductive thin film; bringing the probe of the cantilever close to the biosample;
applying a pulse voltage between the first electrode and the second electrode the thereby allow the fluid to flow out of the fine hole and inject the fluid into the biosample; and
observing change in a surface profile of the biosample through the scanning probe microscope.

2. A biosample manipulation method for dropping a physiologically active substance or a substance for exciting a chemical reaction on a biosample comprising the steps of:
providing the scanning probe microscope with the cantilever comprising a beam portion with its base end supported in a cantilever manner, the probe formed to protrude from the beam portion and having the hollow in itself, the fine hole formed to penetrate from the hollow to an outside of the probe through the probe, and a first electrode and a second electrode provided in the hollow;
filling fluid including the physiologically active substance or the substance for exciting the chemical reaction and having conductivity in the hollow in the cantilever;
bringing the probe of the cantilever close to the biosample;
applying a pulse voltage between the first electrode and the second electrode to thereby allow the fluid to flow out of the fine hole and drop the fluid on the biosample; and
observing change in a surface profile of the biosample through the scanning probe microscope.

3. The biosample manipulation method according to claim 1 and further comprising the step of observing manipulation of the biosample and inner change of the biosample after the manipulation through an optical microscope.

4. The biosample manipulation apparatus according to claim 3, wherein the fluid outflow means comprises a pair of electrodes provided in contact with the fluid and a pulsed power supply for applying a pulse voltage between the respective electrodes.

5. The biosample manipulation apparatus according to claim 1, wherein the beam portion, the probe, and the fine hole constitute a probe of a scanning probe microscope.

6. The biosample manipulation apparatus according to claim 1 and further comprising a manipulation observing means for observing manipulation of the biosample and change of the biosample after the manipulation.

7. The biosmaple manipulation method according to claim 2 and further comprising the step of observing manipulation of the biosample and inner change of the biosample after the manipulation through an optical microscope.

8. The biosample manipulation apparatus according to claim 7, wherein the fluid outflow means comprises a pair of electrodes provided in contact with the fluid and a pulsed power supply for applying a pulse voltage between the respective electrodes.

9. The biosample manipulation apparatus according to claim 2, wherein the beam portion, the probe, and the fine hole constitute a probe of a scanning probe microscope.

10. The biosample manipulation apparatus according to claim 3, wherein the beam portion, the probe, and the fine hole constitute a probe of a scanning probe microscope.

11. The biosample manipulation apparatus according to claim 4, wherein the beam portion, the probe, and the fine hole constitute a probe of a scanning probe microscope.

12. The biosample manipulation apparatus according to claim 7, wherein the beam portion, the probe, and the fine hole constitute a probe of a scanning probe microscope.

13. The biosample manipulation apparatus according to claim 8, wherein the beam portion, the probe, and the fine hole constitute a probe of a scanning probe microscope.

14. The biosample manipulation apparatus according to claim 2 and further comprising a manipulation observing means for observing manipulation of the biosample and change of the biosample after the manipulation.

15. The biosample manipulation apparatus according to claim 3 and further comprising a manipulation observing means for observing manipulation of the biosample and change of the biosample after the manipulation.

16. The biosample manipulation apparatus according to claim 4 and further comprising a manipulation observing means for observing manipulation of the biosample and change of the biosample after the manipulation.

17. The biosample manipulation apparatus according to claim 5 and further comprising a manipulation observing means for observing manipulation of the biosample and change of the biosample after the manipulation.

18. The biosample manipulation apparatus according to claim 7 and further comprising a manipulation observing means for observing manipulation of the biosample and change of the biosample after the manipulation.

19. The biosample manipulation apparatus according to claim 8 and further comprising a manipulation observing means for observing manipulation of the biosample and change of the biosample after the manipulation.
